(19) United States
(12) Patent Application Publication
(10) Pub. No.: US 2014/0273248 A1
(43) Pub. Date: Sep. 18, 2014

(54) Application of Ca Isotope Analysis to the Early Detection of Metastatic Cancer

(71) Applicants: Ariel Anbar, Tempe, AZ (US); Joseph Skulan, Lodi, WI (US); Gwyneth Gordon, Tempe, AZ (US); Rafael Fonseca, Scottsdale, AZ (US)

(72) Inventors: Ariel Anbar, Tempe, AZ (US); Joseph Skulan, Lodi, WI (US); Gwyneth Gordon, Tempe, AZ (US); Rafael Fonseca, Scottsdale, AZ (US)

(73) Assignee: Arizona Board of Regents on behalf of Arizona State University, Scottsdale, AZ (US)

(21) Appl. No.: 14/207,160

(22) Filed: Mar. 12, 2014

Related U.S. Application Data
(60) Provisional application No. 61/784,033, filed on Mar. 14, 2013.

(51) Int. Cl. G01N 33/84

(52) U.S. Cl. CPC G01N 33/84
USPC 436/64

(57) ABSTRACT

Methods using the application of the Ca isotope method for diagnosing and monitoring the progression of cancers that cause bone loss. The methods also can be used for evaluating cancer treatments, such as aromatase inhibitors and other chemotherapeutic agents, for effects on bone density so that treatment can be modified.

### CROSS-REFERENCE TO RELATED APPLICATIONS

This application claims the benefit of U.S. provisional Application No. 61/784,033 filed on Mar. 14, 2013, which is incorporated by reference herein in its entirety.

### STATEMENT REGARDING FEDERALLY SPONSORED RESEARCH

This invention was made with government support under NNX08AQ36G awarded by National Aeronautical & Space Administration (NASA). The government has certain rights in the invention.

### BACKGROUND OF THE INVENTION

At present, the Ca isotope technique is the only method of measuring changes in bone mineral balance (BMB). This technique was originally invented for measuring changes in BMB induced by microgravity and for clinical applications relating to osteoporosis.

Variations in natural Ca isotope composition are a highly sensitive, non-invasive biomarker of bone mineral balance (BMB), providing information on net changes in bone mass that cannot be derived from conventional biochemical markers of bone formation and resorption, on time scales far shorter than those accessible to X-ray techniques like DEXA. The Ca isotopic method has great promise as a prognostic and ultimately a diagnostic biomarker in detecting, monitoring and tailoring effective individual treatments for diseases that involve abnormal loss or gain in skeletal mass, including many cancers.

However, progress in developing the Ca isotope biomarker is impeded by the analytical techniques currently used for high-precision isotope ratio measurements on the most efficient system presently available, multi-collector inductively coupled plasma mass spectrometry (MC-ICP-MS). These techniques were developed mainly for geological applications, which typically require relatively few analyses of inorganic samples. Such techniques have not been optimized for measuring the large number of samples required for application to clinical research and practice. This difficulty is compounded by the fact that high concentrations of organic material in biological samples (e.g., blood and urine) makes processing them much more difficult and time consuming than the processing of inorganic samples, like rocks.

Widespread application of the Ca isotope biomarker thus requires the development of sample preparation and analysis techniques suited to the type and number of samples that will be generated in clinical settings.

### SUMMARY OF THE INVENTION

The embodiments described herein relate to the application of the Ca isotope method to the diagnosing and monitoring the progression of cancers that affect bone. Further embodiments relate to evaluating treatments for cancer that may have an affect on bone density.

These and other aspects of the invention will be apparent upon reference to the following detailed description and figures. To that end, any patent and other documents cited herein are hereby incorporated by reference in their entirety.

### BRIEF DESCRIPTION OF THE DRAWINGS

FIG. 1A-1D depicts data showing a shift in blood Ca isotope composition between patients with monoclonal gammopathy of undetermined significance (MGUS) and multiple myeloma (MM).

FIG. 2 depicts bone mass balance.

FIG. 3 depicts average change during 17 weeks of bed rest from initial values of urinary δ44Ca, and after 17 weeks of bed rest for bone mineral density (BMD) of the femoral neck and lumbar spine, for three treatment groups: alendronate (A), exercise (E), and control (C). For δ44Ca, dark lines inside boxes show 50th percentiles, ends of boxes show 25th and 75th percentiles, and ends of whiskers show upper and lower extremes. For BMD data, central bars show means and ends of whiskers show extreme values.

FIG. 4 depicts the difference in blood δ44/42Ca between patients with monoclonal gammopathy of undetermined significance (MGUS) and multiple myeloma (MM).

FIG. 5 depicts external reproducibility of δ238/235U for 10 aliquots of the standard CRM145 independently processed through chemistry over a 1 week period interspersed with natural samples using the ESI PrepFAST MC. Errors bars indicate the 2sd precision of replicate measurements on a single sample aliquot (N≧3). Dashed lines indicate the 2sd precision for the means of all sample aliquots.

FIG. 6 depicts online measurement of uranium elution using Prepfast system and a quadrupole ICP-MS.

FIG. 7 depicts analytical baseline on Thermo Neptune MCICPMS. The traces of multiple detectors are shown as the magnet is swept across several amu. The baseline is negative because of the charge of the deflected ion beams. Note that an 80 mV change in signal as shown would result in a 200 pptt change on a typical 4 V signal at 44Ca. We achieve a precision of 1 to 2 pptt by a careful combination of extremely stable mass calibration, careful detector placement, precise concentration matching and repeated measurement.

### DETAILED DESCRIPTION OF THE INVENTION

Among other embodiments, two innovations described herein include: validation of an automated system of sample preparation, and optimization of analytical techniques on state of the art MC-ICP-MS instrumentation. Successful implementation of these innovations will increase sample throughput. Thus optimized, a MC-ICP-MS will be fast and efficient enough to support both advanced clinical research and application of the Ca isotope biomarker to clinical practice.

Embodiments described herein relate to the idea that cancer originating in or metastasizing to bone may change the bone mineral balance (BMB) of the skeleton, causing either a net gain or a net loss in bone mass. In addition, treatments for cancer may also have the adverse side effect of causing bone loss. Also, treatments are sometimes aimed at slowing bone loss, but there is no good way to monitor effectiveness. For these reasons there is a clinical need for a method of measuring short-term changes in BMB of cancer patients.

Thus, in one aspect, embodiments of the invention relate to the application of the Ca isotope method to cancer detection and treatment modification. The Ca isotope method is fully described in two publications:

Morgan, J. L., Gordon, G. W., Arrua, R. C., Skulan, J. L., Anbar, A. D., and Bullen, T. D., 2011. High-precision measurement of variations in calcium isotope ratios in urine by multiple collector inductively coupled plasma mass spectrometry. Analytical Chemistry. 83:6956-62.

Morgan, J. L., Skulan, J. L., Gordon, G. W., Romaniello, S. J., Smith, S. M., and Anbar, A. D., 2012. Rapidly assessing changes in bone mineral balance using natural stable calcium isotopes. Proceedings of the National Academy of Sciences, 109:9989-9994, which are hereby incorporated by reference. The method described in these references was used to generate the data of FIGS. 1A, 1B, 1C, and 1D.

The applicability of the Ca isotope method to cancer stems from the fact that some cancers affect the skeleton in ways that alter bone mineral balance (BMB), producing detectable changes in the Ca isotope composition of blood, urine and other biological materials. See, for example, FIG. 1.

Embodiments also relate to the clinical application of the Ca isotope method to cancers where the onset to skeletal involvement marks an important transition in the progress of the disease, the early detection of which may affect both treatment options and prognosis.

Specifically, these embodiments cover:

1. The detection of osteolytic bone lesions that often accompany the transition from monoclonal gammopathy of undetermined significance (MGUS) to multiple myeloma. Osteolytic bone lesions cause a negative shift in BMB that has been detected through Ca isotope analysis of blood.

2. The detection of the metastasis of breast and prostate cancer to bone. This metastasis may involve osteolytic or osteoblastic bone lesions (osteoblastic in prostate cancer, primarily osteolytic in breast cancer). The former leads to bone loss and a negative shift in BMB. The latter may lead to bone gain through hyperostosis, and a positive shift in BMB. Both changes can be detected through Ca isotope analysis of blood, urine or other tissues.

3. The detection of osteolytic or osteoblastic lesions resulting from the metastasis of other cancers to bone. In addition, embodiments cover the application of the Ca isotope method to monitoring changes in bone mineral balance in cancer patients treated with aromatase inhibitors, a known cause of osteoporosis.

A tracerless Ca isotope biomarker allows for the early diagnosis of cancer metastasis to bone, and for detection of osteoporosis caused by aromatase inhibitors. The biomarker is non-invasive and does not require X-rays. There currently are no other technologies that allow for rapid detection of changes in bone mineral balance. Because the technique involves no radiological or other risks, it may be used to continuously monitor patients for bone lesions indicating cancer metastasis or bone loss resulting from cancer treatment. Moreover, quantitatively, the onset of a bone lesion will lead to a larger, faster change than is typical in osteoporosis. In other words, the two would be distinguished by the expected pattern of change in Ca isotopes over time, with metastatic bone cancer being marked by a decrease in BMB over a relatively short time, and age-related osteoporosis causing a slow drop in BMB over a long time.

Natural calcium is a mixture of six isotopes (masses 40, 42, 43, 44, 46, and 48). Calcium isotope compositions conventionally are expressed as δ44Ca ‰, which is the fractional difference, in parts per thousand (‰) between the 44Ca/40Ca ratio of a sample and a laboratory reference material, so that a sample with a δ44Ca of 0‰ has the same isotopic composition as the reference material, while δ44Ca values above and below 0‰ are isotopically heavier and lighter than the reference material, respectively.

Although calcium serves a great many biological functions in the human body, the calcium flux into and out of the skeleton so great that associated fractionation largely controls the calcium isotope composition of soft tissues.

Bone selectively incorporates isotopically light calcium as it mineralizes. As a result δ44Ca of bone calcium is lower than that of soft tissue and of the average δ44Ca of diet. The calcium isotope “fractionation factor” during bone mineralization, which is the instantaneous isotopic difference between newly formed mineral and the soft tissue calcium from which it precipitates, has not been directly measured but is estimated at about −1.3‰.

Calcium isotopes are not fractionated by bone resorption, because osteoclasts dissolve bone mineral in bulk. Isotope fractionation is possible during mineral formation because calcium ions can freely move and compete for sites in growing crystals. However, once incorporated into bone mineral, Ca atoms are locked in a crystal lattice; whether or not they are dissolved depends only on their position in the crystal, and is not affected their mass. Hence calcium returned to soft tissue by bone resorption will have the same isotopic composition as skeletal calcium.

Because calcium isotopes are fractionated by about −1.3‰ during bone formation but are not fractionated during bone dissolution, the soft tissue calcium pool from which bone mineral precipitates is about 1.3‰ higher than bone calcium. In addition, mass balance dictates that when the mass of calcium in the skeleton is constant, the mass and isotope composition of calcium entering and leaving the body will be equal. Thus at ‘steady state’ (where rates of bone formation and resorption are equal) soft tissue and dietary δ44Ca will be equal. Increase in skeletal mass will cause positive excursions in soft tissue δ44Ca, while a decrease in mass will cause negative excursions. These excursions can be detected by mass spectrometric analysis of blood or urine.

The time required for soft tissue δ44Ca to respond to changes in bone mineral balance has not been determined. In the only study of calcium isotopes in humans that has been conducted to date, dramatic changes in urinary δ44Ca were detected in the first samples taken, four weeks after the start of bed rest. But given the short residence times of calcium in soft tissue compartments such changes probably occur in far less than four weeks. It is likely that changes in bone mineral balance will produce detectable shifts in soft tissue δ44Ca on a timescale of days or even hours.

The Ca isotope biomarker employs concepts and techniques developed for geological and environmental science applications that rarely or never have been applied to medicine. Widespread clinical application of the Ca isotope biomarker requires the development of sample preparation and analysis techniques suited to the type and number of samples that will be generated in clinical settings.

Ca isotopes and cancer. A preliminary study conducted with the Mayo Clinic, Scottsdale, δ^{44/42}Ca analysis of blood from 19 patients showed that blood δ^{44/42}Ca was significantly lower in patients with multiple myeloma than in patients with monoclonal gammopathy of undetermined significance (MGUS), an asymptomatic precursor to multiple myeloma (FIG. 4). This difference is consistent with negative BMB caused by bone lesions in multiple myeloma patients. These are single measurements from 19 individuals, uncorrected for background variation in bone δ^{44/42}Ca, and thus represent a worst-case scenario for detecting bone loss using Ca isotopes. In clinical practice, assessing BMB would be accomplished by serial measurements compared to a baseline isotopic value established for each patient.

We expect such results will be possible with other cancers as well because the Ca isotope technique is sensitive enough to detect changes in bone mineral balance likely to be caused by cancerous bone lesions in general. The mathematical model allows changes in soft tissue δ^{44/42}Ca to be quantitatively related to BMB. The drop in urinary δ^{44/42}Ca observed in both of the bed rest studies translates to a net bone loss rate of about 4±1.2%/year, which is consistent with bone loss rates calculated from DEXA measurements. Given current instrumental precision, the lower limit of bone loss detectable by the Ca isotope method is about 2%/year, about four times smaller than the rate of bone loss observed or inferred in multiple myeloma and metastatic breast cancer.

The potential impact on patient care is profound because the Ca isotope technique reveals changes in bone mineral balance at least ten times faster than DEXA, the only other method by which BMB currently can be directly measured. Bone loss is reflected by changes in urinary δ^{44/42}Ca within ten days of the start of bed rest, and after an even shorter interval after the actual start of significant bed-rest induced bone loss, whereas 90 days or more would be required for a rate of bone loss of 4%/year to cause a decrease in bone loss detectable by DEXA.

The relationship between δ^{44/42}Ca and BMB is well enough understood and documented to permit immediate clinical application of the method to situations where baseline measurements are available, and to merit clinical research with large sample suites to validate more widespread applications. The chief obstacle to such work is not theoretical, but technical. Ca isotope analysis of blood and urine currently demands complex multi-stage sample preparation performed by a skilled technician. While superficially similar work is regularly done in clinical laboratories, preparation of biological samples for isotopic analysis requires specialized facilities and expertise that are neither widely available in nor well suited to hospitals and clinics. In particular, the automation and streamlining of sample analysis described below should allow us to have sample turnaround times that will make Ca isotope analysis an important new tool for oncologists.

Isotopic analysis by any method benefits from or requires complete recovery and a relatively pure sample of the element of interest. Complete recovery is necessary because it is essential that the measured isotopic composition be the same as that of the starting material, which cannot be assumed if isotopes of the element are fractionated during sample processing and some of the element to be measured is lost during sample processing. Standard ion exchange chromatography induces isotopic variations larger than the natural variations we wish to measure.

Additionally, most isotope analytical techniques, including the multi-collector inductively coupled plasma mass spectrometry (MC-ICP-MS) that we currently employ for Ca isotope analysis, require extremely high purity. Impurities complicate the mass bias effects during ion transmission into the mass spectrometer that are typically an order of magnitude larger than the natural mass fractionation. To this end, the sample solution has to be as similar as possible to the inorganic standard, since small variations in the relative transmission efficiency between samples and standards are easily on the scale of the natural variations of interest. Interferences, including doubly charged ions and polyatomic ions, are one complication, but matrix effects, including alkali metal concentrations, acid molarity and organics derived from the ion exchange resin, also can cause analytical artifacts that degrade the accuracy and precision of results.

The procedures to deal with these problems have been worked out. Quantitative recovery of high purity Ca is relatively straightforward when dealing with inorganic materials with high Ca concentration such as Ca carbonate or Ca phosphate. A sample is dissolved in acid and Ca isolated using ion exchange chromatography. Modifications or repetitions of this process may be necessary, but achieving required Ca purity usually is not difficult, although it may be time consuming. Purifying Ca from biological materials such as urine and blood is a much more daunting task.

Ca has a high affinity for organic materials (proteins, cell residue etc.—referred to here as “organics”) that are abundant in such samples. These organics must be degraded prior to any other purification step. Removal of organics is a laborious process generally requiring a combination of ashing, microwave digestion and multiple rounds of acid digestion. Sufficient digestion to measure Ca concentrations is insufficient for measuring isotope ratios precisely. Only after organics have been efficiently destroyed is a sample ready for ion chromatography. Two different ion exchange columns are necessary to separate different elements, and a secondary round of acid digestion is necessary to destroy organic residues from the pre-cleaned resins. Determination of quantitative recovery is achieved by measuring the Ca concentration of an aliquot of the sample both prior to and after sample purification.

However, currently all these steps in sample preparation are done manually by highly trained personnel, a process that takes multiple days and adds enormously to the cost and time required for Ca isotope analysis. It also produces unacceptable slow sample turnaround time for routine clinical application. As the time required to process samples is dictated by the chemistry of the samples and the purity requirements of the analytical techniques, it probably cannot be reduced substantially. However, automation of the sample preparation process would greatly increase sample throughput by eliminating the need for a lab technician, allowing 24-hour sample processing, and allowing the simultaneous parallel processing of many samples.

Increased efficiency also could be realized by refining mass spectroscopic techniques. As previously explained, Ca isotope ratios are measured relative to a standard reference material, which maximizes precision and assures inter-lab reproducibility. In theory, a standard would need to be analyzed only once, and the results of all sample analysis compared to that single measure. In practice, because subtle changes in the operating conditions of MC-ICP-MS instruments affect beam stability and cause drift in peak locations, bracketing every sample with a standard before and after is standard practice. Frequency of standard analysis has a large effect on the speed of sample throughput, because time spent analyzing standards is time that cannot be spent analyzing samples. Newer-generation MC-ICP-MS instruments offer more stable operating conditions and better baseline measurements, permitting less frequent standard measurements. In particular, ions from the ⁴⁰Ar or ⁴⁰Ca ion beams can be deflected in older model instruments that lack baffles in the flight tube—resulting in variable and non-zero baselines.

Optimizing sample throughput with MC-ICP-MS can be achieved through improvements in sample processing, and by developing analytical techniques that take advantage of recent technical improvements incorporated into new MC-ICP-MS instruments.

Sample processing. We have validated an existing automated ion exchange system from Elemental Scientific, Inc (Prepfast) for sample purification of U isotopes (FIG. 5). This is the first commercial system to have been validated for metal isotope sample purification. This computer-controlled low-pressure ion exchange chromatography system is configured as an autosampler with four racks of sixty sample positions, four syringe pumps and two six-port valves and a stream selection valve. It has a HEPA-filtered sample enclosure that can be vented to a hood, and capacity for five reagents that can be accurately loaded and dispensed.

This system can be adapted for Ca isotope purification, and modified so that all four steps in sample preparation (sample digestion, purification, recovery determination, and preparation for analysis) can be automated into the same system.

Sample digestion: Our initial sample types, serum and urine, are high in organic matter, including proteins that bind calcium. Our sample purification technique relies on ion exchange chemistry and protein-bound calcium is inefficiently recovered due to poor and variable distribution coefficients. This inefficient recovery can induce isotopic fractionations on the scale of the isotopic variation signals we want to look at. Hence, we need an efficient and highly effective technique for organic degradation. Based on preliminary experiments, high intensity short-wave UV radiation offers the most promise as an in-line system for eliminating organic contaminants prior to sample purification. Samples are loaded into centrifuge tubes in one rack of the Prepfast; while the previous sample was being purified, the following sample would be transferred by a valve in the Prepfast to a quartz tube and subjected to a 1400 W source of UV radiation, with a maximum at 240 nm. This requires an additional multi-port valve to the Prepfast to accommodate the additional digestion location.

Sample purification: Our existing ion exchange chromatography protocol requires several days of active participation by a trained technician, and permits only limited reuse of the ion exchange resin.

Our existing purification techniques utilizes a Biorad AG1X-12 ion exchange resin, followed by an Eichrom Sr-specific resin for separation of Ca and Sr. Our existing purification protocol can use the Biorad resin a maximum of five times before the resin no longer performs adequately. In part because of the required use of concentrated HBr to efficiently separate K from Ca. Ultrapure HBr is expensive, and releases bromine gas, a hazard to lab personnel. The Eichrom resin is not reused because small amounts of elemental carryover can cause significant problems. However, recent technological developments including development of the TODGA ion exchange resin shows significantly better distribution coefficients for Ca.

A significant advantage of the Prepfast system is it can be hooked up in-line to an ICP-MS. The online elution measurements for concentrations using a quadrupole ICP-MS dramatically reduces the development and validation time for a new ion exchange protocols (FIG. 4). In addition, the ability to reuse resin, and use less expensive reagents such as trace metal grade HCl and HNO₃ instead of HBr, will save on analytical costs—an important consideration when scaling up the Ca isotope technique to the level required for clinical research.

Sample recovery determination: In order to ensure that analytical artifacts do not compromise our signal, we must recover over 90% of the Ca and reduce the Sr/Ca ratio to >0.0001, and increase the Ca/(Σalkali metal) to 2. These conditions must be confirmed for all samples, which requires that an aliquot of each purified sample be set aside for elemental concentration measurement. This requirement can be met using the Prepfast system, by loading and automatically diluting a sample aliquot both before and after chemistry. High precision syringe pumps can accurately and repeatedly dispense small volumes. In addition, up to five reagents can be dispensed into four locations. Frequent analysis of in-house standards and spot checks of chemical recovery could be sufficient to ensure quantitative recovery.

Sample preparation for analytical measurement. The Prepfast system can evaporate samples and reconstitute them in acid for MC-ICP-MS measurement. If trace organics from the ion exchange resin are an issue, a second short-wave UV system could be installed for post-chemistry digestion.

Analytical improvements. Analytical efficiency can be improved by reducing the number of replicate measurements of samples and standards required to obtain good sample data. Such reductions can be achieved by improvements in sequence design and baseline measurements that are possible when new MS-ICP-MS instrumentation is combined with the improvements in sample preparation previously discussed.

Improved sequence design. Current techniques use the classic standard-sample-standard measurement technique during MC-ICP-MS analysis. Each sample requires a 60-second uptake time and 60-second wash time. Each analysis requires nearly nine minutes, and each sample is analyzed between three and fifteen times to get acceptable precision. Every five samples, one to two additional secondary standards are analyzed. In addition, each day a series of standards of varying concentration are analyzed, to evaluate the acceptable range of standard-sample concentration matching required for data of adequate quality. This means that only about 40% of the analyses are of actual samples, and nearly 30% of the analysis time is spent either waiting for sample uptake or washout to occur. Hence, less than 30% of the instrument time is spent analyzing samples.

Modifications of the instrumental sequence files may be performed to significantly improve the proportion of time in sample analysis, without sacrificing data quality. The first modification is to eliminate the washout time by using the uptake time of the following sample to rinse out the previous sample, which could produce a 15% improvement in sample throughput. The second modification is to evaluate running two or even three samples bracketed by standards, rather than just one. If the instrument is sufficiently stable, this may still produce acceptable data while potentially doubling sample throughput. Having consistent sample types and residual matrix will be essential to minimize variability in the instrumental mass bias. Hence, this improvement will feed off the improvements in the chemical preparation for consistent sample purity.

The specific sequence of steps we propose for automated preparation of Ca samples in a four-rack PrepFast instrument are as follows:
Sample (urine or serum) put in loading tube (Rack 1)
Sample taken up by probe and transferred to inline quartz capsule, where it is irradiated with short-wave UV. Sample will be irradiated while previous sample is purified.
Syringe pump transfers specified volume of sample (10%) to prechemistry tube (Rack 2).
Sample goes through multi-step ion exchange processing.
Ca eluate is dispensed into collection tube (Rack 3).
Specified volume of sample (10%) transferred to postchemistry vial (Rack 4).
Prechemistry and postchemistry tubes brought to final volume with 0.32 M HNO3.
Ca eluate (Rack 3) dried down and brought up in acid of interest for analytical measurement.
Racks 2 and 4 are transferred onto quadrupole for concentration measurement.

Baseline measurements. Our existing instrument, a Thermo Neptune MC-ICPMS, has been a reliable platform for developing and validating our measurement techniques. However, newer models of this instrument have substantial improvements that would make the measurements easier, more precise and far faster.

Better sensitivity is achieved through the addition of a high-performance scroll pump in place of the rotary pump on our existing model. This scroll pump has better vacuum performance in the front end, enabling better transmission of analyte ions and less dispersion by residual gas pressure.

Because we use a flow rate of 15 L/minute of argon gas to create and maintain the plasma that serves as our ion source, the ion beam at mass ˜40 (⁴⁰Ar and ⁴⁰Ca) is many orders of magnitude larger than our sample. In a straight ion flight tube, this large ion beam can be deflected and reflected, entering the Faraday cups along with the intended sample ion beams. The intensity of the deflected ion beam varies across our nine-detector array, so that even very small differences in the physical position of the detectors cause large differences in intensity of the deflected ion beam reaching each cup (FIG. 5). Our current precision is possible only with a very careful combination of highly stable mass calibration, careful positioning of detectors to minimize the variability of the baselines, precise concentration matching between samples and standards, and repeated sample measurements (up to fifteen times). Newer generation instruments have baffles in the flight tube that eliminate the problem with deflected ion beams. The instrument manufacturer does not offer a retrofit addition of baffles to existing machines, so we are unable to correct the design flaws that limit our analytical precision on our current instrument.

We anticipate that far fewer replicates will be required to produce our current precision, given an instrument with installed baffles. For other metal mass-dependent isotope systems, typical samples are measured in triplicate with a precision of 1 pptt or better. Achieving this efficiency with Ca would more than double our sample throughput.

Several modifications to the Prepfast ion exchange chromatography system are required to implement our improvements to the Ca isotope method for clinical samples. These include: 1) addition of another multi-port valve to incorporate the quartz tube needed for sample digestion 2) integration of short-wave UV radiation source for sample digestion 3) incorporation of a heater block in the eluent rack to allow evaporation of samples to dryness and 4) modifications to the system's software to allow computer control of these processes.

Specific steps that will be taken to implement each innovation are given in the table below.

TABLE 1

| Innovation | Task |
|---|---|
| Sample preparation: | Procure a hollow quartz tube and evaluate how much short wave length |
| Sample digestion | UV radiation is necessary for efficient sample digestion. Criterion for |
| | successful digestion is to reduce the sample (serum and urine) |
| | absorbance to the same level as our current protocol for microwave |
| | digestion and repeated hot plate digestions. May require a cooling |
| | system and pressure release valve to prevent catastrophic failure. |
| | Higher temperature and pressure is likely to improve digestion if |
| | system can tolerate those conditions. An existing 1400 W Newport |
| | solar simulator with short-wavelength UV transmitting mirrors can be |
| | used during testing. Digestion may also require addition of mineral |
| | acid such as nitric acid to enhance organic degradation. |
| Sample digestion | Validate that UV-digested samples produce isotope data of similar |
| | quality to our existing protocols. Validation will require that at least 50 |
| | samples are processed using both digestion protocols. |
| Sample purification | Evaluate elution curves with TODGA resin online with Q-ICP-MS. |
| | Optimize for quantitative Ca separation and maximum removal of |
| | critical interfering elements including Na, K, Mg, Ti and Sr. Evaluate |
| | eliminating secondary Eichrom Sr-specific resin. Criterion for |
| | successful evaluation will be quantitative recovery greater than 95%, |
| | and elemental ratios required for purity as outlined in Morgan et al |
| | (Analytical Chemistry paper). |
| Yield determination | Measure calcium recovery during purification by comparing the pre- |
| | chemistry and post-chemistry purification process. This step would be |
| | to determine if pre-chemistry and post-chemistry aliquots can be |
| | accurately and sufficiently precisely diluted to calculate chemical |
| | recovery within 10%. Criterion for determining this will be to directly |
| | compare the yield recovery for both volumetric aliquots and dilutions to |
| | gravimetrically determined manual aliquots and dilutions. Both serum |
| | and urine samples will be tested over multiple concentrations to |
| | determine the acceptable range of conditions for yield determination |
| | within 10%. |
| | Evaluate robustness of chemical purification and determine if |
| | measuring chemical recovery is required for every sample. An |
| | alternate method would be to measure chemical recovery on a specified |
| | percentage of samples (5%, 10% or 25%). This would be buttressed by |
| | regular monitoring of procedural blanks and in-house standards in each |
| | batch of samples purified. Systematic analysis to determine how many |
| | standards and blanks are required in each sample purification suite, and |
| | if the analytical order should be randomized or a regular, intermittent |
| | addition to the purification sequence. |
| Sample preparation for | Evaluate using inert polymeric support resin (Eichrom Prefilter |
| isotopic measurement | support) to remove organics post-ion chromatography. These organics |
| | routinely require a secondary nitric acid/hydrogen peroxide digestion |
| | for good quality MC-ICPMS data. Criterion for evaluation will be to |
| | reduce the UV absorption spectra of samples to a similar level to that of |
| | our current protocol. A secondary criterion will be sample analysis |
| | with a similar reproducibility and accuracy to our current configuration. |
| | Validate protocol for drying down samples in the Prepfast rack, and |
| | then rediluting them with 0.32M nitric acid for isotopic measurement. |
| Analytical | Evaluate using take-up time of one sample to also serve as wash time |
| Improvements: | for previous sample, and see if sequence timing needs to be adjusted. |
| Instrumental sequence | Criterion for determining acceptable performance is the standard |
| improvements | deviation of replicate analyses of samples can not degrade by more than |
| | 5%. Criteria period will be at least 48 hours of instrument run time. |
| Instrumental sequence | Evaluate using two samples between bracketing standard. Criterion |
| improvements | for determining acceptable performance is the standard deviation of |
| | replicate analyses of samples can not degrade by more than 5%. |
| | Criteria period will be at least 48 hours of instrument run time. |
| Instrument performance | Systematic documentation of sensitivity and baseline improvements |
| improvements | with new instrumentation. |
| | Evaluation of increased analysis throughput due to superior |
| | instrumentation; requires running at least five days of the same |
| | samples with the same sequence configuration on both instruments. |

Hydroxylapatite precipitation experiments. There are a number of published techniques for precipitating pure (stoichiometric) hydroxylapatite. Many of these techniques use concentrated reagents and relatively high temperature (>80° C.) to quickly and efficiently produce hydroxylapatite. Other techniques are intended to mimic conditions of bone mineralization, and produce hydroxylapatite at physiological temperature and pH. In the former experiments hydroxylapatite generally precipitates directly from solution; the initial precipitate from physiological solutions generally is a poorly crystalline material that matures to hydroxylapatite over a period of hours to days. Measurements of Ca isotope fractionation during both types of hydroxylapatite precipitation will reveal the sensitivity of fractionation to the physical conditions of formation. High (ca 80-90° C.) temperature methods that produce hydroxylapatite after little aging will be used to explore the dependence of Ca isotope fractionation on temperature, pH and precipitation rate; precipitation under CO₂ free but otherwise physiological conditions will be used to measure Ca isotope fractionation between Ca²⁺ and the initial poorly crystalline precipitate, and changes in the Ca isotope composition of this precipitate as it matures into stoichiometric hydroxylapatite.

Carbonate and Mg substitution experiments. Bone mineral is not pure hydroxylapatite, but typically contains substitutions, the most common of which are percent-level substitutions of Mg²⁺ for Ca²⁺ and CO₃²⁻ for PO₄³⁻. Possible effects of such substitution on Ca isotope composition of hydroxylapatite will be investigated by precipitating Mg and CO₃-rich under physiological conditions. If Ca isotope fractionation is found to be substitution-dependent, follow-up experiments will be conducted to quantify this dependence and constrain its effect on the Ca isotope composition of natural bone mineral. The degree of Mg and CO₃ substitution in hydroxylapatite is easily manipulated by varying [Mg²⁺] in the precipitating solution and PCO₂ in the air above the solution.

Hydroxylapatite-collagen experiments. Natural bone mineral is precipitated on a protein (primarily collagen) matrix, which accounts for up to 40% of the mass of mature bone. It is generally believed that collagen is laid down first, and is subsequently mineralized with hydroxylapatite. The presence of collagen could affect Ca isotope fractionation by, for example, affecting the diffusion of Ca²⁺ to crystallization sites within the collagen matrix. It is possible to replicate this process in vitro, by abiotically precipitating hydroxylapatite on collagen sponge obtained by demineralizing bone. It also is possible to co-precipitate collagen and hydroxylapatite from the same solution. Both of these experimental approaches will be used to determine whether, and how, the presence of collagen affects the Ca isotope composition of co-existing bone mineral. In addition, because it most closely resembles natural bone mineral, hydroxylapatite precipitated with collagen is used in the isotope exchange experiments described next.

TABLE 2

| | | | | | | | Replicates | |
|---|---|---|---|---|---|---|---|---|
| Experiment | | | Isotope | | | Samples/ | or | Total |
| type | Conditions | Mineral | comp. | Variables | Duration | variation (1) | variations | sample |
| Precipitation | High T, pH | Hydroxylapatite | Natural | pH, T, | Hours | 3 | 10 | 30 |
| | | | | precipitation rate | | | | |
| Precipitation/ | Physiological | Amorphous Ca | Natural | Mineral phase | hours | 7 (2) | 10 | 70 |
| exchange | CO₂ free | phosphate, | | | to | | | |
| | | hydroxyalapatite | | | days | | | |
| Substitution | Physiological | Amorphous Ca | Natural | Mineral phase, | hours | 7 | 4 | 28 |
| | | phosphate, low Ca | | CO3 and Mg | to | | | |
| | | carbonate | | presence/absence | days | | | |
| | | hydroxyalapatite | | | | | | |
| Substitution | Physiological | Amorphous Ca | Natural | Mineral phase, | hours | 7 | 20 | 140 |
| | | phosphate, low Ca | | degree of CO3 | to | | | |
| | | carbonate | | and Mg | days | | | |
| | | hydroxyalapatite | | substitution | | | | |
| Precipitation/ | Physiological | Hydroxyalapatite | Natural | Co-precipitation of | hours | 3 | 8 | 28 |
| exchange | | | | hydroxyalapatite | to | | | |
| | | | | and collagen | days | | | |
| Precipitation/ | Physiological | Hydroxyalapatite | Natural | Re-mineralization | hours | 3 | 2 | 6 |
| exchange | | | | of de-meneralized | to | | | |
| | | | | collagen | days | | | |
| Exchange | Physiological | Amorphous Ca | Labeled | Mineral phase | hours | 3 | 8 | 28 |
| | | phosphate maturing | | | to | | | |
| | | to low Ca carbonate | | | days | | | |
| | | hydroxyalapatite | | | | | | |
| Long term | Physiological | Low Ca carbonate | Labeled | Time | hours | 40 (3) | 3 (4) | 120 |
| exchange | high T | hydroxyalapatite | | | to | | | |
| | | | | | years | | | |

Table 2:
Notes:
(1) each experiment produces three samples: initial solution, final solution, and final mineral. An additional pair of samples (solution and mineral) are generated for each time point sampled between the initial and final samples.
(2) sampled at two intermediate points during the maturation of the initial poorly crystalline precipitate into hydroxylapatite.
(3) Sampled at ten time points, from ca 1 hour to 1 year.
(4) One each of labeled solution + unlabeled mineral, unlabeled solution + labeled mineral, and unlabeled solution + unlabeled mineral. Total number of samples: 494.

Isotope exchange experiments. The possibility that equilibrium isotope fractionation affects the Ca isotope composition of hydroxylapatite is investigated in experiments that most closely replicate in vivo conditions—high Mg, high CO₃ hydroxyapatite produced in the presence of collagen under physiological conditions. Equilibrium isotope fractionation between a mineral and the solution from which it precipitates requires isotopic exchange between solution and mineral. The rate of isotopic exchange can be measured using a ⁴⁸Ca tracer, in parallel experiments in which solution containing an isotopically labeled solution is incubated with an unlabeled precipitate, and an unlabeled solution is incubated with a labeled precipitate. ⁴⁸Ca will be used as a tracer because it is abundant enough to be available in relatively pure form (unlike ⁴³Ca and ⁴⁶Ca), yet scarce enough that very high enrichments (ca. 1000%) can with much less material than would be required for more abundant isotopes

The rate of isotopic equilibration is determined by measuring the change over time in tracer concentration in solution and precipitate in both experiments. Isotopic exchange rates will be measured during two phases of the hydroxylapatite precipitation experiments: during the maturation of hydroxylapatite from the initial poorly crystalline precipitate, and between precipitating solution and mature hydroxylapatite.

Experimental design and data. Table 1 summarizes the experimental suite. This plan may be modified based on emerging results. For example, if Ca isotope fractionation during the precipitation and maturation of pure hydroxylapatite is found to be the same as in the precipitation of highly CO₃ and Mg substituted hydroxylapatite, there will be no need for a more detailed investigation of the effects of CO₃ and Mg substitution.

In order to constrain mass balance, precipitation experiments will be done using batch, rather than continuous processes. Constant temperature and pH will be maintained. To the extent possible, precipitation reactions will be phosphate-limited so that only a relatively small fraction of available Ca²⁺ precipitates, which will minimize isotope distillation effects. Ca isotope compositions of starting solutions, ending solutions and precipitates will be measured. Mineralogy of precipitates will be determined using a combination of XRD, SEM and TEM. Except for the long-term exchange experiments, experiments in which initial precipitates are aged will be conducted in duplicate, permitting separate measurements of initial precipitates and precipitates after aging. Precipitates will be separated from solution by centrifuge, washed several times, and dried prior to isotopic, mineralogical and crystallographic analysis. Experiments will be scaled to produce a minimum of 100 ug of hydroxylapatite precipitate, enough material to meet all analytical demands. Ca isotope fractionation factors will be computed from the difference in δ^{44/42}Ca between initial solution and bulk precipitate, using a Rayleigh distillation model as necessary to correct for distillation effects.

Hydroxylapatite for long term exchange experiments will be co-precipitated in three batches, each large enough to produce 1000-1500 mg of precipitate. One batch will be spike with ⁴⁸Ca to an enrichment of about 1000%, the other two batches will be unspiked. 20 aliquots of ca 50 ug of spiked hydroxyapatite will be mixed with 20 aliquots of unspiked solution from one of the two unspiked batches and vice versa. 20 aliquots of mixed solution and hydroxylapatite from the second unspiked batch also will be taken. All aliquots will be placed in sealed containers and continuously agitated at a constant temperature between 70 and 80° C. (high enough to significantly accelerate isotopic exchange without affecting the stability of hydroxylapatite). Aliquots from each of the three experiments (spiked hydroxylapatite+unspiked solution, unspiked hydroxylapatite+spiked solution, unspiked hydroxylapatite+unspiked solution) will be harvested in pairs over the course of a year (or more). The spiked experiments will measure the rate of isotopic equilibration, if it can be measured on the timescale of the experiments. The unspiked experiment will provide data needed to calculate the Ca²⁺-hydroxylapatite equilibrium fractionation factor. In addition to mineralogical analyses, crystal size and morphology will be monitored during long term precipitation, to gain information on the mechanism of any observed equilibration, and how the rate of equilibration relates to crystal size.

Analytical methods. In order to prevent interferences from degrading data quality, hydroxylapatite samples are acid digested followed by ion exchange purification. To ensure accurate results, quantitative recovery during column chemistry and efficient matrix removal, is verified by quadrupole ICP-MS measurement of aliquots before and after chemistry.

Isotope abundances will measured by multiple-collector (MC) ICP-MS (ThermoScientific Neptune). Samples are introduced into a 1200-W plasma with a desolvating sample introduction system (Apex, CPI International) in medium-mass resolution. 30 measurement cycles are collected for each analysis; ratios of the ion beams ⁴⁴Ca/⁴²Ca,⁴⁴Ca/⁴³Ca,⁴⁸Ca/⁴²Ca, and ⁴⁸Ca/⁴³Ca are calculated and averaged into a single measurement. Standard-sample-standard bracketing is used to correct for instrumental mass bias. Secondary standards, purified through chemistry and measured by a different lab with thermal ionization mass spectrometry (TIMS), are run every few samples to ensure measurement reproducibility and accuracy over time. The bracketing standards used for all samples and external standards are the in-house standard, National Institute of Standards and Technology Ca ICP solution (NIST lot no. X-10-39Ca).

The abundance ratio of δ^{44/42}Ca can be measured using <25 μg of Ca with a typical precision of ±0.2 (±2 SI)) ‰ compared to the standard.

All samples will be characterized with SEM to determine crystal size, size distribution and morphology. Secondary electron imaging on the FEI XL30 environmental FESEM will provide spatial resolution of a few nanaometers for detailed analysis of crystal morphology and sample homogeneity. EDX will be used to assist in phase identification and test for chemical heterogeneity. FESEM will also be used to image collagens and their structural relationship to hydroxylapatite. FIB-SEM will be employed to image mineral-collagen aggregates in 3D by slicing into aggregates and imaging the sliced surfaces. This technique can be used for 3D tomography, if needed and for imaging of the apatite-collagen interface. TEM will be used to further characterize the structure of the hydroxylapatites in the run products, employing one of several analytical TEM instruments in the LE-CSSS. TEM imaging combined with selected-area electron diffraction or nanodiffraction will be used to determine crystallinity, crystallographic orientations and fabrics in physiological samples.

HRTEM imaging is particularly useful for characterizing the maturation process from amorphous to crystalline material and the crystal defects that may result from precipitation or maturation. Sample preparation for TEM will depend on the size of the particles synthesized. If the particles are 10s of nanometers in size, the samples can be imaged directly in TEM after dispersal onto holey carbon support grids. If the crystals are larger than this, they will have to be thinned for analysis. The simplest method is crushing followed by dispersal on a grid so that thin particle fragments can be images. Larger grain-sizes and apatite-collagen aggregates will be sectioned using the FIB lift-out techniques. This sections provide ideal samples for TEM imaging, diffraction and EDX microanalysis. FIB section orientation can be selected to investigate specific crystal orientations or fabric directions.

The claims are not intended to be limited to the embodiments and examples described herein.

1. A method for the detection of cancer-related osteolytic and osteoblastic bone lesions, comprising:
analyzing a bodily fluid of a patient through Ca isotope analysis; and
detecting whether said fluid indicates a rate of change in bone mineral balance indicative of osteolytic and osteoblastic bone lesions.

2. The method of claim 1, wherein said fluid is blood.

3. The method of claim 1, wherein said analyzing is performed through multi-collector inductively coupled plasma mass spectrometry.

4. The method of claim 1, wherein said bodily fluid is treated with high intensity short-wave UV radiation prior to purification.

5. A method for the detection of the metastasis of breast or prostate cancer to bone, comprising the steps of:
analyzing a bodily fluid of a patient having breast or prostate cancer through Ca isotope analysis; and
detecting whether said fluid indicates any decrease or increase in bone mineral balance based on said Ca isotope.

6. The method of claim 5, wherein said increase or decrease is indicative of bone metastasis of breast cancer or prostate cancer.

7. The method of claim 5, wherein said fluid is blood.

8. The method of claim 5, wherein said bodily fluid is treated with high intensity short-wave UV radiation prior to purification.

9. A method for monitoring changes in bone mineral balance in cancer patients, comprising:
analyzing through Ca isotope analysis a bodily fluid of a patient being treated with one or more anti-cancer drugs or therapies; and
detecting whether said fluid indicates a change in bone mineral balance.

10. The method of claim 9, wherein said one or more anti-cancer drugs or therapies comprises aromatase inhibitors.

11. The method of claim 9, wherein said fluid is blood.

12. The method of claim 9, wherein said bodily fluid is treated with high intensity short-wave UV radiation prior to purification.
